(19) United States
(12) Patent Application Publication
(10) Pub. No.: US 2014/0008827 A1
(43) Pub. Date: Jan. 9, 2014

(54) Colored Putty Compositions and Methods of Use

(71) Applicant: Robert B. Hankins, del Valle, TX (US)

(72) Inventor: Robert B. Hankins, del Valle, TX (US)

(21) Appl. No.: 13/543,853

(22) Filed: Jul. 8, 2012

(51) Int. Cl. A61K 6/02; A61C 13/08

(52) U.S. Cl. 264/20; 106/35

(57) ABSTRACT

Putty compositions having colorants incorporated therein to provide materials useful in a dental laboratory or operatory for a variety of functions including manufacture of dental restorations, the invention facilitates inter alia the staining of the restorations to desired shades for maximum cosmetic effect.

### TECHNICAL FIELD

The invention relates to compositions of matter and methods of use thereof for manufacture of dental restorations to facilitate the production of desired shadings of the restorations to produce desirable cosmetic effects.

### BACKGROUND ART

Putty materials, often referred to as peg putties, have long been used in the dental laboratory and increasingly in the dental operatory in the manufacture of dental restorations. Within the last several years, the inventor of the putty material disclosed herein introduced into the market a white putty material comprised of refractory ceramic fibers, and particularly vitreous aluminosilicate fibers, this material in certain forms being previously and primarily used in the building industry as a caulking disposed about stove pipes and the like to space the pipes from combustible materials forming walls and the like. This refractory ceramic fibrous material introduced by the inventor has been and continues to be widely adapted by others in the dental arts for particular use as a “peg putty” in the manufacture of dental restorations. This white peg putty is placed, for example, in bridges and on veneers and the like with a peg or pin inserted into the putty prior to firing to harden the putty. The bridge or veneer, that is, the restoration, can then be easily handled while the restoration is stained, glazed or subjected to any other appropriate operation.

As such, the white refractory ceramic fibrous material, manufactured for use as a caulking material by Unifrax of Niagara Falls, N.Y., has been adequate for industry-wide use for a number of years but has been improved according to the present invention by the coloring of the refractory ceramic fibrous material to various shades of color that relate to shade guides commonly used in the dental industry. This coloring of the putties of the invention produce a variety of shades allowing duplication of tooth color in a resulting restoration more easily and more precisely especially in the staining of the restoration.

The prior art has also contemplated various procedures for determining color and shade for tooth and dental restorations. As one example, U.S. Pat. No. 6,743,014 of Kerschbaumer et al discloses shade determination apparatus and methodology useful in the practice of clinical dentistry and in the dental laboratory to permit a tooth or a dental restoration to be colored and/or shaded for aesthetic purposes by allowing ease of reproducibly matching coloration and translucence with teeth that will be adjacent to the tooth or to the restoration being fabricated. Pruden, in U.S. Pat. No. 6,623,271, describes fabrication of a dental restoration whereby a dental restorative material is compared to shade guide components under the lighting conditions present in the oral cavity. In U.S. Pat. No. 6,499,998 Kerschbaumer et al describe reference templates useful in the shade determination of a tooth or dental restoration so that comparison of the article being fabricated to a patient's teeth can be accomplished.

Accordingly, the issued United States patents noted above inter alia illustrate the attention previously devoted in the industry to matching the coloration and shading of prosthetic teeth and dental restorations to existing teeth with which such restorations function. The present invention provides compositions and method of use of such compositions that provide continued advancement in the art of fabrication of prosthetic teeth and dental restorations so that an aesthetically pleasant appearance is imparted to the dentition of the individual for whom a restorative article is intended.

### SUMMARY OF THE INVENTION

The invention provides peg putty materials having a variety of colors or shades that relate to shade guides used in the dental industry, one or more of the colored putty materials being employed in the same manner as prior uncolored peg putties in manufacture of a restoration but which improve such use by allowing a practitioner to more effectively stain or otherwise shade the restoration to duplicate the natural tooth color of the individual patient in the restoration. Use of the colored peg putty materials of the invention typically involves selection of one colored material from a set of differently colored materials to color match a stump having a color matching a particular patient's teeth. Use of colored peg putties of the invention facilitates shading of restoration by minimizing the time necessary to appropriately shade the restoration. The present colored peg putty materials can be used with all prior materials commonly employed for manufacture of restorations including porcelains, zirconia and ceramic materials as well as lithium silicates and disilicates including monolithic species thereof and with processes employed not only in a dental laboratory but also in the dental operatory whether conventional or with computer-aided techniques.

Accordingly, it is a primary object of the invention to provide putty materials capable of use for insertion of a peg or pin thereinto to mount dental restorations and the like, the present putty materials being colored to a variety of shades relative to conventional and convenient shade guides to facilitate appropriate shading of the restoration through conventional processes such as staining and the like.

It is another object of the invention to provide all-in-one putty materials and a stump guide for facilitating the staining of ceramic restorations in particular and especially highly translucent restorations, one advantage of the invention being the provision of peg putty materials in tooth-colored shades.

It is a further object of the invention to provide colored putty materials that can be placed inside a veneer, crown or the like to replicate tooth color, the color of the peg putty being chosen to attain such replication, the peg putty also facilitating the mounting of veneers and the like during a staining process without the need for pre-drying and which is easily removed after firing.

It is yet another object of the invention to provide colored putty materials useful for healing of cracked or broken pressables, PVC elements, jacket crowns, veneers and the like as well as for procedures including soldering, mounting of copings on pegs allowing for little or no lingual band and for staining and reglazing of veneers and the like to maintain marginal and internal integrity.

It is a still further object of the invention to provide a colored putty material useful for securing virtually all types of restorations on pegs while firing and which can be used as a stump shade inside restorations and particularly ceramic restorations that can be fired directly on a putty material of the invention.

Further objects and advantages of the invention will become more readily apparent in light of the following description of the invention.

### DESCRIPTION OF EMBODIMENTS

The putty materials of the invention are composed in preferred embodiments of refractory ceramic fibers and particularly vitreous aluminosilicate fiberts such as are available in materials manufactured and marketed by Unifrax of Niagara Falls, N.Y. under the trade name of Fiberfrax Pumpable GS Insulating Mix, components of the Unifrax composition including water in a range of 50 to 70 percent by weight; refractories, fibers and aluminosilicates in a range of 20 to 25 percent by weight; silica, particularly amorphous silica, in a range of 5 to 10 percent by weight and hydroxyethylcellulose in a range of one to 3 percent by weight.

A preferred material is selected with a weight percent of silica more near the lower portion of the stated range. The melting point of the preferred refractory ceramic fibrous material is approximately 1760° C. and the specific gravity of the fibrous material is within the range of 2.50 to 2.75. This material provides a particularly useful base material although similar materials produced by other manufacturers are also acceptable as base materials.

The fibrous material thus described is well known, toxicological and other data being available in a Material Safety Data Sheet MSDS No. M0132 effective Mar. 5, 2007 and available from Unifrax.

Compositions according to the invention that are resistant to freezing can also be formulated. Such compositions typically utilize water in the lower part of the weight percent range noted above, particularly 50 percent water by weight and further include 5 to 10 percent by weight of ethylene glycol or 10 to 20 percent by weight of propylene glycol. These weight percentages are exemplary only. Compositions so formulated are considered to be freeze stable.

The fibrous material referred to herein has previously been introduced into the dental field by the present inventor for use as a peg putty inter alia that is useable with porcelain and other systems for healing of cracked or broken pressables, PVC elements, jacket crowns, veneers, etc. This previously introduced material can be used in procedures including soldering, for mounting copings on pegs allowing for little or no lingual band and during the staining and re-glazing of veneers and the like to maintain marginal and internal integrity. This prior fibrous material is bright white in color.

Preferred embodiments of the putty materials of the invention are formed with the refractory ceramic fibrous material described above as a base material but with the addition of selected colorants to provide a series of colored putty materials matched to conventional and convenient shade guides allowing a practitioner to select at least one of a series of the differently colored putty materials in order to duplicate tooth color of a particular patient so that a restoration can be appropriately colored or stained with reduction of time necessary to produce an appropriately colored restoration.

Colorants can include conventional food coloring as well as other preferably non-toxic colorants. In the formation of ivory-colored putty, for example, one drop of an ivory colorant formed of yellow, brown and white food coloring can be added to approximately twelve cubic centimeters of white refractory ceramic fibrous material. Various shades of the present putty materials can be formed by similar mixtures of colors such as food colorings with drop-wise addition to a white refractory ceramic fibrous material to form a desired color shade matched to a dental color guide of choice. It is to be understood that the refractory ceramic fibrous material can be a color other than white when employed as a base material, colors such as off-white or cream being acceptable. Other base materials can also be employed in a practice of the invention, such materials being chosen for an ability to hold a peg or pin, for example, so that a restoration can be held and manipulated without the need to manually touch the restoration. A patty of the putty can also be used for manual manipulation in place of a peg or pin. A desirable base material must be capable of being manually or otherwise removed from a restoration after firing and prior to use.

The colored putty materials of the invention can be used in a manner and for uses essentially identical to the use of the white putty formed of vitreous aluminosilicate fibers inter alia as has been previously used in the dental arts after color selection of the putty. The present colored putty materials are typically chosen for use to approximate the coloration or shade of a stump guide or a tooth guide for example, which is to receive a restoration such as a crown.

Base materials useable within the environment of the invention can be comprised of materials other than refractory ceramic fibrous materials. A material marketed by Ivoclar under the trade name of Object Fix is a composition of aluminum oxide, quartz, water and hydroxyethylcellulose. This cream-colored material can be placed in or on a restoration followed by insertion of a peg or pin for mounting the restoration for subsequent operations thereon. This material can be colored according to the invention in a manner such as are preferred embodiments of the invention are colored to yield benefits similar to those characterized by use of the preferred embodiments.

Other base materials can be colored according to the invention, such materials having the ability to set up much in the manner of plaster of Paris include a mixture of quartz, cristobalite, ammonium phosphate and magnesium oxide. Another base material can be formed of silicon dioxide, magnesium oxide, aluminum oxide and ammonium phosphate. A further base material suitable for coloration according to the invention and useful in a manner similar to use of preferred embodiment of the invention can be formed of quartz, feldspar and silicon dioxide. These base materials typically require mixing and a short period of time to set up before a peg or pin can be inserted into these materials.

Putty placed on a restoration and receiving a peg or pin permits handling of the restoration through the agency of the peg. The peg is positioned in the putty to avoid contact with any portion of a restoration such as a veneer to prevent the color of the peg from being visible through the restoration. The restoration can then be stained, glazed or otherwise treated without resort to use of an additional composite stump to attain the correct shade. After staining or the like, the restoration is conventionally glazed and heat treated followed by removal of the hardened putty which has hardened after any firing step. In essence, procedures conventional to a dental laboratory or operatory are followed.

A series of the present colored putty materials can be packaged as a kit with colors typically useful to a practitioner, such a kit optionally including other materials useful to a practitioner.

Examples of the colored putty materials contemplated according to the invention are described as individual formulations hereinafter, the several examples being referenced by product designations for convenience of description, it being understood that a virtually infinite number of color selections are possible with the formulations provided hereinafter being exemplary only with specific formulations being chosen for utility in actual practice of the invention. Units of measure noted in the formulations are defined by standard designations of measure as are common in the industry. However, for ease of consideration of the units of measure, sixty (60) drops are equivalent to one dram; 29,5735 cubic centimeters are equivalent to one ounce; and 128 ounces are equivalent to one gallon, one gallon therefore being equivalent to 3785 cubic centimeters. Further, since the putty materials of the invention are typically provided in syringes containing twelve (12) cubic centimeters of putty material, it is to be noted that approximately 315 syringes can be filled with one gallon of putty material, the material weighing approximately eight (8) pounds.

### EXAMPLE I

### Shade HL1

The putty material of this formulation is ivory in color and is formed by the addition of 4.25 drams of an ivory colorant such as is formed of a combination of yellow, brown and white food colors to one gallon of base material such as the refractory ceramic fibrous material noted herein, the ivory colorant alternatively being a food coloring produced by Jack Guttman, Inc. of West Chester, Ohio under the product designation Ivory 136BOB, IP-5228. The food colorings described hereinafter are similarly products of Jack Guttman, Inc., and are noted herein by the formal name of the coloring as used by this source. Similar food colorings available from alternate sources are satisfactory as are other colorants of differing type, desirable characteristics of such colorants including low or no toxicity, the ability to mix with a chosen base material and the ability to burn away along with the base material when necessary.

### EXAMPLE II

### Shade HL2

The putty material is formulated by the addition of seven (7) drams of the Ivory colorant noted in Example 1 together with 21 drops of Nut Brown food color per one (1) gallon of base material.

### EXAMPLE III

### Shade HL3

The putty material is formulated by the addition of seven (7) drams of the Ivory colorant noted in Example 1 together with 45 drops of Lemon Yellow food color, one (1) dram of Nut Brown food color and twenty (20) drops of Red food color per one (1) gallon of base material.

### EXAMPLE IV

### Shade HL4

The putty material is formulated by the addition of six (6) drams of the Ivory colorant noted in Example I together with 36 drops of Flesh food color, 2.25 drams of Nut Brown food color, 3.3 drams of Lemon Yellow and twenty (20) drops of Red food color respectively per one (1) gallon of base materials.

### EXAMPLE V

### Shade HL5

The putty material is formulated by the addition of eight (8) drams of the Ivory colorant noted in Example I together with ten (10) drams of Nut Brown, six (6) drams of Flesh, 1.2 drams of Green and four (4) drops of Lemon Yellow food colors respectively per one (1) gallon of base material.

### EXAMPLE VI

### Shade HL6

The putty material is formulated by the addition of 4.2 drams of the Ivory colorant noted in Example I together with 36 drops of Nut Brown, 36 drops of Flesh, 36 drops of Lemon Yellow and eighteen (18) drops of Facial food colors respectively per gallon of base material.

### EXAMPLE VII

### Shade HL7

The putty material is formulated by the addition of seven (7) drams of the Ivory colorant noted in Example I together with one (1) dram of Nut Brown food color per gallon of base material.

### EXAMPLE VIII

### Shade HL8

The putty material is formulated by the addition of seven (7) drams of the Ivory colorant noted in Example I together with thirteen (13) drams of Nut Brown and 1.3 drams of Green food color respectively per gallon of base material.

### EXAMPLE IX

### Shade HL9

The putty material is formulated by the addition of seven (7) drams of the Ivory colorant noted in Example I together with ten (10) drams of Nut Brown, six (6) drams of Dark Fleshtone, 1.2 drams of Green and four (4) drops of Lemon Yellow food colors respectively per gallon of base material.

Colorants such as food colors available from other producers of such materials can also be used according to the invention, such colorants being either organic or inorganic and preferably non-toxic. Stains common in the industry and such as are available from the Vident Corporation under the trade name Vita can also be used to color base materials according to the invention.

In use, an acceptable color or shade of putty is selected through comparison to a tooth guide or stump guide in light of the coloration of the teeth of an individual for whom a restoration or the like is to be prepared. The selected putty is injected into the restoration in a manner essentially identical to the use of prior stump putty materials. The restoration is then placed on a firing peg and stained in a conventional manner. Without the need for drying, the stained restoration is conventionally baked. After baking, the dried putty is conventionally removed.

It is to be understood that formulations other than as are explicitly detailed herein can be employed in a practice of the invention without departing from the scope of the invention, the scope of the invention being set forth according to the recitations of the appends claims.

1. A composition of matter useful as a putty material in the fabrication of dental restorations and the like, comprising a base material suitable for use as a peg putty or the like and a colorant when formulated with the base material matches a shade of dentition in the mouth of a tooth or teeth present in the mouth.

2. The composition of matter of claim 1 wherein the base material is a refractory ceramic fibrous material.

3. The composition of matter of claim 2 wherein the base material comprises a refractory ceramic fibrous material having vitreous aluminosilicate fibers.

4. The composition of matter of claim 1 wherein the base material comprises a white or off-white material.

5. The composition of matter of claim 1 wherein the base material comprises water in a range of 50 to 70 percent by weight; refractories, fibers and aluminosilicates in a range of 20 to 25 percent by weight, silica in a range of 5 to 10 percent by weight and hydroxyethylcellulose in a range of one to three percent by weight.

6. The composition of matter of claim 5 wherein silica comprises amorphous silica.

7. The composition of matter of claim 1 wherein the base material comprises a mixture of aluminum oxide, quartz, water and hydroxyethylcellulose.

8. The composition of matter of claim 1 wherein the base material comprises a mixture of silicon dioxie, magnesium oxide, aluminum oxide and ammonium phosphate.

9. The composition of matter of claim 1 wherein the base material comprises a mixture of quartz, feldspar and silicon dioxide.

10. The composition of matter of claim 1 wherein water is present in the base material in a range of 50 to 70 percent by weight and an antifreeze material is present in a weight percent suitable to cause the composition of matter to be freeze stable.

11. The composition of matter of claim 1 wherein the colorant comprises at least one food color.

12. The composition of matter of claim 11 wherein the colorant comprises a mixture of yellow, brown and white food colors to form an ivory colorant.

13. The composition of matter of claim 12 wherein 4.25 drams of the colorant is admixed with one gallon of the base material.

14. The composition of matter of claim 13 wherein the base material comprises a refractory ceramic fibrous material.

15. The composition of matter of claim 1 wherein the colorant is selected from the group consisting of the ivory colorant, brown food color, yellow food color, red food color, flesh food color, fleshtone food color, green food color and facial food color.

16. The composition of matter of claim 15 wherein the base material comprises a refractory ceramic fibrous material.

17. A method for formulation of a putty material useful as a peg putty in the fabrication of dental restorative elements comprising the steps of admixing a colorant with a base material to form a putty having a color similar to a possible shade of dentition in the mouth of a tooth or teeth present in the mouth.

18. The composition of matter of claim 17 wherein the base material comprises a refractory ceramic fibrous material.

19. The composition of matter of claim 18 wherein the colorant comprises an admixture of yellow, brown and white food colors to form an ivory colorant.

20. The composition of matter of claim 19 wherein the colorant in quantity of 4.25 drams is admixed with one gallon of the base material.

21. The composition of matter of claim 20 wherein the colorant is selected from the group consisting of the ivory food colorant, brown food color, yellow food color, red food color, flesh food color, fleshtone food color, green food color and facial food color.

22. A method for using a putty to produce dental restorations and the like, comprising the steps of:
a) selecting a putty from a group of putty materials of differing coloration to match a patient's teeth as indicated by a tooth guide or a stump guide;
b) utilizing the selected putty to mount the restoration;
c) stain or otherwise treat the restoration; and
d) without drying, bake the treated restoration.
